(19) United States
(12) Patent Application Publication
(10) Pub. No.: US 2010/0136125 A1
(43) Pub. Date: Jun. 3, 2010

(54) METHOD OF TREATING INFLAMMATORY BOWEL DISEASE

(75) Inventors: David P. Jacobus, Princeton, NJ (US); Guy Alan Schiehser, Washington Crossing, PA (US); Kathy Lynne Ales, Princeton, NJ (US); Laura Ransom Jacobus, Princeton, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Jacobus Pharmaceutical Company, Inc., Princeton, NJ (US)

(21) Appl. No.: 12/520,923

(22) PCT Filed: Dec. 26, 2007
(86) PCT No.: PCT/US07/26350

§ 371 Date: Feb. 18, 2010

Related U.S. Application Data
(60) Provisional application No. 60/882,403, filed on Dec. 28, 2006.

(51) Int. Cl. A61K 9/50; A61K 31/606; A61K 9/16; A61P 1/06

(52) U.S. Cl. 424/497; 514/166; 424/400; 424/489; 424/490; 424/494

(57) ABSTRACT

Methods are disclosed for treating patients suffering from inflammatory bowel disease, including ulcerative colitis or Crohn's disease, by administering an oral or enema dosage form containing at least one aminosalicylate active ingredient. Granular dosage forms and kits are also disclosed.

### CROSS REFERENCE TO RELATED APPLICATIONS

This application claims the benefit of U.S. Application No. 60/882,403 filed Dec. 28, 2006, the disclosure of which is incorporated herein by reference.

### FIELD OF THE INVENTION

The present invention generally relates to methods of treating patients suffering from inflammatory bowel disease, including ulcerative colitis or Crohn's disease, by administering an oral or enema dosage form containing at least one aminosalicylate active ingredient.

### BACKGROUND OF THE INVENTION

Inflammatory bowel disease (IBD) can involve either or both the small and large bowel. Ulcerative colitis and Crohn's disease are the best known forms of IBD and both fall into the category of “idiopathic” inflammatory bowel disease because their etiology is unknown. Pathologic findings are generally not specific. “Active” IBD is characterized by acute inflammation. “Chronic” IBD is characterized by architectural changes of crypt distortion and scarring. Crypt abscesses (active IBD with neutrophils in crypt lumens) can occur in many forms of IBD not just ulcerative colitis.

Ulcerative colitis is a chronic inflammatory and ulcerative disease arising in the colonic mucosa, characterized most often by bloody diarrhea. The rectum is almost always involved and though the disease may remain localized, it may extend into the colon rarely involving the entire colon. The course of the disease generally involves periods of remission and exacerbation (flairs). Extraintestinal symptoms, particularly arthritis, may occur and long-term risk of colon cancer is high. Treatment is with 5-ASA, corticosteroids, immunomodulators, anticytokines, antibiotics, and occasionally surgery. The etiology for ulcerative colitis is unknown.

Crohn's disease is chronic, episodic, inflammatory condition of the gastrointestinal (GI) tract characterized by transmural inflammation and skip lesions. Crohn's disease can affect any area of the GI tract, from the mouth to the anus, but it most commonly affects the ileum. The inflammation can cause pain and can make the intestines empty frequently, resulting in diarrhea. Crohn's disease is an intermittent disease with acute flares, spontaneous remissions, a significant placebo response and no therapy which is fully satisfactory (Su C, Lichtenstein G R, Krok K, Brensinger C N, Lewis J D. A Meta-Analysis of the Placebo Rates of Remission and Response in Clinical Trials of Active Crohn's Disease. Gastroenterology 2004; 126:1257-1269).

There is wide variability in the reported incidence of Crohn's disease in the general population. Data for the United States provide incidence rates ranging from 3.6 (Kurata J H, Kantor-Fish S, Frankl H, et al. Crohn's Disease among ethnic groups in a large health maintenance organization. Gastroenterology 1992; 102:1940) to 8.8 (Nunes G C, Ahlquist R E, Jr. Increasing incidence of Crohn's disease. Am J Surg 1983; 145:578) cases per 100,000. The current US population is estimated at almost 300,000,000 (http://www.census.gov/cgi-bin/popclock), suggesting that in the United States alone there are between 10,650 and 26,032 new cases of Crohn's disease diagnosed each year. The prevalence of 105.7 (Sedlack R E, Whisnant J, Elveback L R, et al. Incidence of Crohn's disease in Olmsted County, Minnesota, 1935-1975. Am J Epidemiol 112: 759-63, 1980) per 100,000 suggests that 312,682 people in the United States are living with Crohn's disease today. Between 30% (Mekhjian H S, Switz D M, Melnyk C S, Rankin G B, and Brooks R K. Clinical Features and Natural History of Crohn's Disease. Gastroenterology. 1979; 77:898-906; Rijk M C M, van Hogezand R A, van Lier H J J and van Tongeren J H M. Sulphasalazine and Prednisone Compared with Sulphasalazine for Treating Active Crohn Disease. Annals of Internal Medicine. 1991; 114 (6):445-450) and 42% (Singleton J W, Hanauer S B, Gitnick G L, Peppercorn M A, Robinson M G, Wruble L D, Krawitt E L, and the Pentasa Crohn's Disease Study Group. Mesalamine Capsules for the Treatment of Active Crohn's Disease: Results of a 16-Week Trial. Gastroenterology. 1993; 104:1293-1301) of Crohn's disease patients have disease limited to the small bowel whereas between 37% and 74% have ileocecal involvement (Schmidt C, Fels T, Baimeister B and Vetter H. Current Medical Research and Opinion. 1996; 13 (7):4176-425). It is estimated that 75% of new cases occur in patients over the age of 20 (Mendeloff A I. The epidemiology of inflammatory bowel disease. Clin Gastro 1980:9:259-270) and very few new cases occur after the age of 65. Flares cannot be predicted and the number of flares per year is highly variable, both between and within patients.

There are a number of current treatments for Crohn's disease, including aminosalicylates, systemic steroids such as prednisone and budesonide, immunosuppressives such as azathioprine (AZA, Imuran), 6-mercaptopurine (6-MP), and methotrexate, and TNF antagonists, such as infliximab. In addition, in certain cases atypical medications such as thalidomide can be used. However, each of these current treatments presents challenges. For example, aminosalicylates, including 4-aminosalicylic acid, sulfasalazine, and its metabolite mesalazine (5-aminosalicylic acid), which were dosed at low levels of 1.5 g/day (slow release), did not induce or maintain remission (Schreiber S, Howaldt S, Raedler A. Oral 4-aminosalicylic acid versus 5-aminosalicylic acid slow release tablets. Double blind, controlled pilot study in the maintenance treatment of Crohn's ileocolitis. Gut 1994 August: 35 (8):1081-5). Systemic steroids and immunosuppressives have dose/duration-related toxicities. Budesonide only provides a brief benefit. (Sandborn W J, Sands B E, Colombel J-F, Abreu M T. New and Emerging Roles for TNF Antagonists in Managing Crohn's Disease: An Expert Panel Discussion. Scientific Frontiers, Inc, Medscape for WebMD Release date: Aug. 4, 2006, page 10). The TNF antagonists exhibit loss of efficacy, infusion reactions, need for concomitant immunosuppression, and the need for commitment to “indefinite” maintenance dosing. (See Sandborn, pages 40 to 54).

4-Aminosalicylic acid (para-aminosalicylic acid) is sold in the U.S. by Jacobus Pharmaceutical under the trademark PASER®. 4-Aminosalicylic acid is an antibacterial used to treat tuberculosis. It has been used for over forty years in the treatment of IBDs. It is thought to act via NF-κB (Nuclear Factor Kappa B) inhibition and free radical scavenging. However, as briefly discussed above, both 4-aminosalicylic and 5-aminosalicylic acid and its prodrug, as they are currently administered, do not effectively treat Crohn's disease.

Multiple studies have been conducted in an attempt to demonstrate that aminosalicylates are effective in Crohn's disease and aminosalicylates are frequently prescribed as maintenance therapy, despite uncertain efficacy (Hanauer S B and Dassopoulos T. Evolving Treatment Strategies for Inflammatory Bowel Disease. Annu. Rev. Med. 2001; 52:299-318). Nevertheless, no U.S. Food and Drug Administration (FDA) approval has yet been granted for aminosalicylates in the treatment of Crohn's disease.

Based on the dose responses to aminosalicylates in distal ulcerative colitis, the failures of previous studies to convincingly demonstrate effectiveness in Crohn's disease are to be expected. The dosage and local release rates (dosage/time relationship) of the 5-ASA preparations studied to date in Crohn's patients are generally far below the topical levels required for response in patients with distal ulcerative colitis. Furthermore, the lesions of Crohn's disease are deeper and much more penetrating than the lesions in ulcerative colitis, often resulting in abscesses, peritonitis, and fistulae. Thus, the questionable efficacy of aminosalicylates in Crohn's disease is primarily or at least in part due to insufficient dosing and hence inadequate delivery of active agent to affected tissues (Singleton J W, Hanauer S B, Gitnick G L, Peppercorn M A, Robinson M G, Wruble L D, Krawitt E L, and the Pentasa Crohn's Disease Study Group. Mesalamine Capsules for the Treatment of Active Crohn's Disease: Results of a 16-Week Trial. Gastroenterology. 1993; 104:1293-1301).

Therefore, it would be desirable to develop methods and oral and enema dosage forms that are effective in treating a patient suffering from inflammatory bowel disease without causing immunosuppression and by delivering sufficient quantities of an active drug to the affected target tissues over a sufficient period of time In addition, said methods and oral and enema dosage forms would be used therapeutically at a specific phase of the disease process (onset and duration of an acute flare) and for a limited amount of time so as to maximize short term relief, minimize long term progression of the disease and minimize long term toxicological and other deleterious effects. While low dose level maintenance therapy may be acceptable, the method described in the present invention demands high dose and focused drug delivery during periods of acute flares for effective short and long term benefit. The methods and dosage forms of the present invention are directed toward these, as well as other, important ends.

### SUMMARY OF THE INVENTION

Accordingly, the present invention is directed, in part, to novel methods and oral and enema dosage forms for administering aminosalicylate active ingredients to treat inflammatory bowel disease (IBD), including ulcerative colitis and Crohn's disease and other related diseases, by interrupting and effectively treating the acute flares. The methods of the invention involve administering the active ingredient for a minimum period to achieve maximum control of the disease process. Because most patients will be back to pre-flare status, further treatment will not be required, but may be continued but is preferably not continued, as described further below. Among the benefits of the methods of the invention are sparing the patient of disease activity without immunosuppression and other side effects. The benefits of prompt control of acute flares extend beyond relief of the pain and diarrhea often associated with IBD. The longer the acute flares persist the more likely the disease process will progress and result in serious complications. Thus, prompt remission of acute flares will have a beneficial effect on the course of the ulcerative colitis, Crohn's disease and other inflammatory bowel diseases, even without continued administration or maintenance of the active ingredients.

Delayed-release granular 4-aminosalicylic acid product, PASER®, is approved by the United States Food and Drug Administration to be used in dosages of up to 4 grams three times daily for treatment of multi-drug resistant tuberculosis. Given the outstanding safety profile of aminosalicylates, the higher local doses for treatment of ileocecal Crohn's disease is a long-awaited advance in the treatment of this debilitating disease. Developing the full capability of aminosalicylates is important even as new classes of “biologic agents” are introduced and tested (Egan L J, Sandborn W J. Advances in the Treatment of Crohn's Disease. Gastroenterology 2004; 126:1574-1581). Effective at higher topical doses in the small intestine and proximal colon, aminosalicylates will limit or delay the need to resort to other more toxic and costly medical modalities and/or surgery.

Accordingly, in one aspect, the invention is directed to methods of treating inflammatory bowel disease in a patient, comprising the step of:

administering orally or by enema to said patient for a treatment period sufficient to control acute flares of said inflammatory bowel disease a solid dosage form comprising:

a therapeutically effective amount of an aminosalicylate active ingredient selected from the group consisting of 4-aminosalicylic acid, 5-aminosalicylic acid, a pharmaceutically acceptable salt thereof, or a combination thereof;

a first rate-controlling polymer that forms a pH labile enteric coating for administration of said solid dosage form orally;

optionally, a first rate-controlling polymer that forms a pH labile enteric coating for administration of said solid dosage form by enema;

wherein said dosage form releases drug at a therapeutically effective rate and in a region of the intestinal tract of said patient where said acute flares are present.

In another aspect, the invention is directed to granular dosage forms, comprising:

generally spherical particles comprising:

an active ingredient selected from the group consisting of 4-aminosalicylic acid, 5-aminosalicylic acid, a pharmaceutically acceptable salt thereof, or a combination thereof;

a first rate controlling polymer that forms a pH labile enteric coating; and

at least one second rate-controlling polymer;

wherein said particles have a diameter of about 1.3 mm to about 3.5 mm; and

wherein said dosage form releases in USP 23-NF 18:

less than about 5%, by weight, of said active ingredient over 2 hours at pH 1.0; and

about 40%, by weight, to about 60%, by weight, of said active ingredient in about 1 hour at pH 7.2.

In another aspect, the invention is directed to kits, comprising:

instructions for administering an oral dosage form to a patient suffering from inflammatory bowel disease;

the granular oral dosage form described above; and

at least one oral delivery vehicle.

In other aspects, the invention is directed to kits, comprising:

instructions for administering an enema to a patient suffering from inflammatory bowel disease;

the granular enema dosage form described above; and

at least one enema delivery vehicle.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 shows the results of in vitro dissolution studies of various aminosalicylates at simulated gastric pH over 2 hours: PASER® 4-ASA is compared with PENTASA® ER 5-ASA 500 mg tablets, PENTASA® CR 5-ASA 250 mg and 500 mg capsules, and ASACOL® 5-ASA 400 mg.

FIG. 2 shows comparative in vitro dissolution studies of PASER® 4-ASA, PENTASA® 5-ASA, and ASACOL® 5-ASA at pH 7.5 after 2 hours in simulated gastric pH.

FIG. 3 shows in vitro dissolution studies of PASER® 4-ASA at pH 6.8 and pH 7.2.

### DETAILED DESCRIPTION OF THE INVENTION

As employed above and throughout the disclosure, the following terms, unless otherwise indicated, shall be understood to have the following meanings.

Abbreviations used herein include:
Ac-PAS—Acetyl-para-aminosalicylate
ASA—Aminosalicylic acid
4-ASA—4-aminosalicylic acid (also known as PAS or para-aminosalicylic acid)
5-ASA—5-aminosalicylic acid
CD—Crohn's disease
CDAI—Crohn's Disease Activity Index
HBI—Harvey Bradshaw Index
IBD—Inflammatory Bowel Disease (Includes Crohn's Disease, ulcerative colitis and other related diseases
mCDAI—Modified Crohn's Disease Activity Index
MDR-TB—Multidrug resistant tuberculosis
6MP—6-Mercaptopurine
PCDAI—Pediatric Crohn's Disease Activity Index
6TG—6-Thioguanine
TPMT—Thiopurine methyl-transferase

As used herein and in the appended claims, the singular forms “a,” “an,” and “the” include the plural reference unless the context clearly indicates otherwise.

As used herein, the term “control” as used in connection with “acute flares” means at least a statistical significant improvement in the score in the Crohn's Disease Activity Index, Harvey-Bradshaw Index, Inflammatory Bowel Disease Questionnaire, inflammatory marker (including (1) hemoglobin (2) the erythrocyte sedimentation rate (ESR); (3) C-reactive protein (CRP); and (4) fecal calprotectin), or a combination thereof.

As used herein, “controlled release”, “delayed release,” or “extended release,” used interchangeably herein refers to deliver of the active drug ingredient to the target organ in delayed, controlled and gradual manner relative to an immediate release formulation. Suitable means to achieve controlled release include that use (1) diffusion controlled (membranes and matrices); (2) chemically controlled (erosion and pendent chain), and (3) solvent activated (osmotic pressure and swelling), and specifically include reservoir devices, monolithic devices (matrix devices), microspheres, microencapsulation, pendent devices, enteric films, pH dependent coatings, osmotically controlled devices, electrically stimulated release devices, hydrogels, chemical prodrugs including polymeric covalent substrate attachments and intercalation of active drug molecules between the layers of a layered inorganic host (such as layered double hydroxides).

As used herein, “side effect” refers to a consequence other than the one(s) for which an agent or measure is used, as the adverse effects produced by a drug, especially on a tissue or organ system other then the one sought to be benefited by its administration. In the case, for example, of agents for the prevention or treatment of neuron loss or neurological disorders, the term “side effect” may preferably refer to such conditions as, for example, immunosuppression, infusion reactions, and toxicity.

As used herein, “therapeutically effective amount” refers to an amount of the active ingredient as described herein that may be effective to inhibit, or treat the symptoms of the acute flares of IBD, or to prevent, inhibit, or diminish the symptoms of the acute flares of IBD. In general, the therapeutically effective amount of the active ingredients of the invention, which are administered orally or by enema, range from 4 grams per day to 12 grams per day in divided or single doses.

As used herein, “treating” refers to the preventative, curative, and palliative treatment of a condition, and includes, in particular, not only the prevention and/or treatment of a condition per se, but also the prevention of the progression and frequency of the acute flares associated with IBD.

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Thus, the term “base addition salt” refers to the corresponding salt derivative of a parent compound that has been prepared by the addition of a base. The pharmaceutically acceptable salts include the conventional salts or the quaternary ammonium salts of the parent compound formed, for example, from inorganic or organic bases. For example, such conventional salts include, but are not limited to, those derived from inorganic bases such as lithium hydroxide, sodium hydroxide, potassium hydroxide, calcium hydroxide, magnesium hydroxide and ammonium hydroxide and the salts prepared from organic amines, such as methyl amine, ethyl amine, isopropyl amine, piperidine, piperizine, pyrrolidine, ethanolamine, morpholine, diazapine, ethylene diamine, pyridine, quinoline, quinuclidine, and the like.

All forms of the active ingredients, including free acid, free base, and zwitterions, are contemplated to be within the scope of the present invention. The free base/free acid and the sodium, potassium, and calcium salts are the preferred forms of the aminosalicylate active ingredients of the invention. The free acid/free base and the sodium salts of the aminosalicylate active ingredients are especially preferred.

As used herein, “prodrug” is intended to include any covalently bonded carriers that release the active parent drug or whose form is converted in vivo when such prodrug is administered to a patient. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Specifically included among the active ingredients useful in the methods and oral dosage forms of the invention is olsalazine and balsalizine; prodrugs of 5-aminosalicylic acid, Prodrugs are also meant to include the covalent attachment of aminosalicylates to polymers wherein covalent bond is cleaved chemically or enzymatically to release free drug to the affected region.

As used herein, “patient” refers to an animal, including a mammal, preferably a human. As used herein, “adult” refers to a human at least 18 years of age. As used herein, “child” refers to a human less than 18 years of age.

### 5-Aminosalicylates in Inflammatory Bowel Disease:

Aminosalicylates have a long history in the treatment of inflammatory bowel disease (IBD). Since the discovery that 5-aminosalicylic acid (5-ASA) represents the therapeutic moiety of sulfasalazine, 5-ASA has been administered either rectally in the form of suppositories, enemas or foam, or orally in different delayed- or controlled-release forms with the development of a variety of azo prodrugs (Klotz U. The Role of Aminosalicylates at the Beginning of the New Millennium in the Treatment of Chronic Inflammatory Bowel Disease. European Journal of Clinical Pharmacology. 2000; 56:353-362). Rectal administration of 5-ASA in active ulcerative colitis results in remission rates of between 40% and 75% (Klotz U. The Role of Aminosalicylates at the Beginning of the New Millennium in the Treatment of Chronic Inflammatory Bowel Disease. European Journal of Clinical Pharmacology. 2000; 56:353-362). A dose-dependent response to orally administered 5-ASA has been demonstrated in mildly to moderately active ulcerative colitis (d′Albasio G, Paoluzi P, Campieri M, Porro G B, Pera A, Prantera C, Sturniolo G C, Miglioli M, and The Italian IBD Study Group. Maintenance Treatment of ulcerative proctitis with mesalazine suppositories: A double-blind placebo-controlled trial. Am J Gastroenterol 1998; 93 (5):799-803; Marakhouski Y, Fixa B, Holomán J, Hulek P, Lukas M, Bátovský, Rumyantsev V G, Grigoryeva G, Stolte M, Vieth M, Breinwald R, & The International Salofalk Study Group. A double-blind dose-escalating trial comparing novel mesalazine pellets with mesalazine tablets in active ulcerative colitis. Aliment Pharmacol Ther 2005; 21:133-140), and maintenance of remission in patients with ulcerative proctitis or colitis also appears to be dose-dependent (D′Albasio G, Pacini F, Canarri E, Messori A, Trallori G, Bonanomi A G, Bardazzi G, Milla M, Ferrero S, Biagini M, Quartanta S, and Amorosi A. Combined therapy with 5-aminosalicylic acid tablets and enemas for maintaining remission in ulcerative colitis: A randomized double-blind study. Am J Gastroenterol 1997; 92 (7):1143-1147; Paoluzi O A, Iacopini F, Rica R, Crispino P, Marcheggiano A, Consolazio M, Rivera M, Paoluzi P. Comparison of two different daily dosages (2.4 vs 1.2 g) of oral mesalazine in maintenance of remission in ulcerative colitis patients: 1-year follow-up study. Aliment Pharmacol Ther 2005; 21:1111-1119).

5-Aminosalicylates in Crohn's Disease:

The reports concerning effectiveness of 5-ASA preparations in active Crohn's disease have been conflicting. Nevertheless, as with ulcerative colitis, the data suggest a dose response5 and it has been stated that “to be effective, 5-ASA should be given as a daily dose above 3 g” (Klotz U. The Role of Aminosalicylates at the Beginning of the New Millennium in the Treatment of Chronic Inflammatory Bowel Disease. European Journal of Clinical Pharmacology. 2000, 56:353-362). This statement is based on the findings of a 16-week trial of controlled-release mesalamine in active Crohn's disease: remission (>50 decrease in CDAI with CDAI<151 at final visit) occurred in 43% of patients in the 4 g group, 24% in the 2 g group and 23% in the 1 g group, compared with 18% in the placebo group. Therapeutic benefit (decrease of 50 points or more in CDAI) occurred in 39% in the 2 g group and 36% in the 1 g, compared with 40% in the placebo group. These data suggest that a 1 g or 2 g daily dose of aminosalicylates is no different than placebo (Singleton J W, Hanauer S B, Gitnick G L, Peppercorn M A, Robinson M G, Wruble L D, Krawitt E L, and the Pentasa Crohn's Disease Study Group. Mesalamine Capsules for the Treatment of Active Crohn's Disease: Results of a 16-Week Trial. Gastroenterology. 1993; 104:1293-1301). By contrast, one gram four times daily produced a 43% remission rate and a 64% therapeutic benefit at 6 weeks (Singleton J W, Hanauer S B, Gitnick G L, Peppercorn M A, Robinson M G, Wruble L D, Krawitt E L, and the Pentasa Crohn's Disease Study Group. Mesalamine Capsules for the Treatment of Active Crohn's Disease: Results of a 16-Week Trial. Gastroenterology. 1993; 104:1293-1301). Thus, it would appear that a daily dose of 4 g of oral 5-ASA may be the minimum effective dose.

A three-armed Italian trial in mild to moderate Crohn's disease compared (1) a daily dose of 4 g of microgranular 5-ASA (ASACOL microgranular [GF 1930-IT], a gelatin capsule with microgranules coated with Eudragit S) with (2) 4 g of 5-ASA in a tablet form (coated with Eudragit S) and (3) methylprednisolone 40 mg for 2 weeks followed by a tapering schedule (Prantera C, Cottone M, Pallone F, Annese V, Franze A, Cerutti R, Porro G B, and a Cooperative Study Group. Mesalamine in the Treatment of Mild to Moderate Active Crohn's Ileitis: Results of a Randomized, Multicenter Trial. Gastroenterology. 1999; 116:521-526). This trial included patients with ileal and cecal disease as well as patients with colonic aphthous ulcers, but excluded concomitant steroid therapy. At 3 weeks, microgranular 5-ASA resulted in a 61% remission rate compared with 46% who received 5-ASA as a tablet and 61% in the steroid group. The patients who entered had mild disease (Crohn's Disease Activity Index or CDAI 204-257) and there was no placebo arm. With placebo remission rates in active Crohn's disease reported as high as 49% (Wright J P, Jewell D P, Modigliani R, et al. for the International Organization for the Study of Inflammatory Bowel Disease (IOIBD) (1995) A randomized double-blind, placebo-controlled trial of olsalazine for active Crohn's disease. Inflamm Bowel Dis 1995; 1:214-216), the absence of a placebo arm in this study makes it difficult to assess the magnitude of the benefit. One in 28 patients who received the 5-ASA microgranular formulation developed acute pancreatitis which resolved upon withdrawal from the drug. The specific preparation used in this trial is not available in the United States, although a different microgranular 5-ASA preparation (PENTASA®) is available. No claims are made about the effectiveness of this product for active Crohn's disease in the labeling and it is not FDA-approved for the treatment of Crohn's disease.

The utility of 5-ASA in maintaining remission in Crohn's disease is also questioned. Heterogeneity of patients, preparations and dosages certainly contributes to this uncertainty. Of note, yet again, a dose-dependent response has been reported in the post-operative setting (Frieri G, Pimpo M T, Andreoli A, et al. Prevention of post-operative recurrence of Crohn's disease requires adequate mucosal concentration of mesalazine. Aliment Pharmacol Ther 1999; 13:577-582). Furthermore, the importance of adequate dosing in Crohn's disease has now entered the medical therapy literature with recommendations that PENTASA 5-ASA (controlled-release capsule) be used as “4 g daily in four divided doses,” (Dhillon S, Loftus E V. Medical Therapy of Crohn's Disease. Current Therapy Options in Gastroenterology 2005, 8:19-30) and clinical trials assessing efficacy of different formulations of 5-ASA in doses up to 6 g/d are ongoing.

Adverse Effects of 5-Aminosalicylates:

The primary side-effects of 5-aminosalicylates include nausea, vomiting, dizziness, headache, and abdominal pain, with rare hypersensitivity reactions (e.g. pericarditis, pancreatitis, bronchospasm, exacerbation of colitis) (Hanauer S B, Stathopoulos. Risk-Benefit Assessment of Drugs Used in the Treatment of Inflammatory Bowel Disease. Drug Safety 1991; 6 (3):192-219). The incidence of nephrotoxicity in patients taking 5-aminosalicylate therapy is estimated to be on the order of one in 4000 patients per year, with most cases occurring in patients who were on treatment for longer than 12 months (Muller A F, Stevens P E, McIntyre A S, Ellison H, Logan R F. Experience of 5-aminosalicylate nephrotoxicity in the United Kingdom. Aliment Pharmacol Ther 2005; 21:1217-1224). No correlation was noted between 5-aminosalicylate dose and degree of renal impairment and all 5-aminosalicylate preparations were implicated.

In vitro kinetic studies have demonstrated that sulfasalazine as well as 3-, 4-, and 5-ASA are non-competitive inhibitors of thiopurine methyltransferase (TPMT), with IC₅₀ values of 78, 99, 2600 and 1240 μM, respectively (Szulmanski C L, Weinshilboum R M. Sulphasalazine inhibition of thiopurine methyltransferase: possible mechanism for interaction with 6-mercaptopurine and azathioprine. British Journal of Clinical Pharmacology 1995; 39 (4):456-459). As a non-competitive inhibitor of thiopurine methyltransferase (TPMT), olsalazine in combination with 6-mercaptopurine has been associated with bone marrow suppression in an individual with low levels of thiopurine methyl-transferase (TPMT) activity (Lewis L D, Benin A, Szumlanski C L, Otterness D M, Lennard L, Weinshilboum R M, Nierenberg D W. Olsalazine and 6-mercaptopurine-related bone marrow suppression: a possible drug-drug interaction. Clinical Pharmacology and Therapeutics 1997; 62 (4):464-45). No such reports have surfaced to date with regard to 4-ASA.

### 4-Aminosalicylates in Inflammatory Bowel Disease:

### 4-Aminosalicylates in Ulcerative Colitis:

There has been surprisingly little work involving 4-ASA in IBD, particularly in the United States. However, in 1984, a 4-aminosalicylic acid rectal preparation was shown to have equal activity to the 5-ASA preparation (Campieri A, Lanfranchi G A, Bertoni F, Brignola C, Bazzocchi G, Minguzzi M R, and Labo G. A Double-Blind Clinical Trial to Compare the Effects of 4-Aminosalicylic Acid to 5-Aminosalicylic Acid in Topical Treatment of Ulcerative Colitis. Digestion. 1984; 29:204-208). 4-ASA enemas were shown to be more effective in inducing clinical and sigmoidoscopic improvement than placebo at either 1 or 2 gram doses for distal ulcerative colitis (Selby W S, Bennett M K, Jewell D P. Topical treatment of distal ulcerative colitis with 4-amino-salicylic acid enemas. Digestion 1984; 29:231-234). Subsequently, a 2 gram nightly 4-ASA retention enema was shown to result in significant improvement in clinical, sigmoidoscopic and histologic variables after 8 weeks in active left-sided ulcerative colitis (83% 4-ASA vs 15% placebo, p<0.005) (Ginsberg A L, Beck L S, McIntosh T M, et. al. Treatment of left-sided ulcerative colitis with 4-aminosalicylic acid enemas. A double blind placebo-controlled trial. Ann Intern Med 1988 February; 108 (2):195-9). A small two week randomized placebo controlled trial in distal ulcerative colitis compared 1 g 4-ASA bid and 2 g 4-ASA bid to placebo retention enemas bid, and reported no significant difference between the groups with regard to symptoms or sigmoidoscopic appearance, although the 2 g dose but not the 4 g dose appeared to be more effective than placebo with regard to specific symptoms (number of bowel movements, amount of mucus in stools, sense of urgency) even at 2 weeks (Gandolfo J, Farthing M, Powers G, Eagen K, Goldberg M, Berman P, Kaplan M. 4-Aminosalicylic acid retention enemas in treatment of distal colitis. Digestive Disease and Sciences. 1987; 32 (7):700-704). With more prolonged treatment using 2 g bid in open-label follow-up, 46% of participants had complete resolution of symptoms and sigmoidoscopic abnormalities by 8 weeks, with an additional 31% experiencing improvement (Gandolfo J, Farthing M, Powers G, Eagen K, Goldberg M, Berman P, Kaplan M. 4-Aminosalicylic acid retention enemas in treatment of distal colitis. Digestive Disease and Sciences. 1987; 32 (7):700-704). Another study showed that 6 gram 4-ASA enemas were more effective than placebo in mild to moderate ulcerative colitis extending more than 60 cm above the anus (Beeken W, Howard D, Bigelow J, et al. Controlled trial of 4-ASA in ulcerative colitis. February 1997 Dig Dis Sci 42 (2):354-8). Especially noteworthy was the use of 4-ASA enemas in three patients with 5-ASA-induced acute pancreatitis. 4-ASA proved both safe and effective, with control of the symptoms of inflammatory bowel disease and no recurrence of pancreatitis (Daniel F, Seksik P, Cacheux W, et al. Tolerance of 4-aminosalicylic acid enemas in patients with inflammatory bowel disease and 5-aminosalicylic-induced acute pancreatitis. Inflamm Bowel Dis 2004 May 10; 30:258-260). Thus, it appears that 4-ASA and 5-ASA may be interchangeable in the treatment of ulcerative colitis. 4-ASA may even be the preferred agent as it has not been associated with pancreatitis. There are also suggestions that 4-ASA may be more effective than corticosteroids (O′Donnell L J D, Arvind A S, Hoang P, Cameron D, Talbot I C, Jewell D P, Leonard-Jones J E, Farthing M J G. Double blind, controlled trial of 4-aminosalicylic acid and prednisolone enemas in distal ulcerative colitis. Gut 1992; 33:947-949; Halphen M, Chevrel B. Traitement des formes distales de la Recto-colite hémorragique par le 4-ASA Na. Med. Chir. Dig—Actualités Thérapueutiques. 1998; 27:329-333 (from AFA website)). A 4-ASA enema (Norgine Pharma's Quadrasa®) dosed at 2 g/d has been commercially available in Europe since 2000 and is significantly better tolerated then 5-ASA enemas (Marteau P and Halphen M. Étude comparative ouverte randomisée de l′ efficacité et de la tolérance de lavements de 2 g d′acide 4-amino-salicylique (4-ASA) et de 1 g d′acide 5-amino-salicylique (5-ASA) dans les formes basses de rectocolite hémorragique. Gastroenterol Clin Biol, 1995, 19, 31-35).

4-Aminosalicylic Acid in Crohn's Disease:

No studies have been reported to date of 4-ASA in the treatment of acute or active Crohn's disease. However, one German trial compared 1.5 g/d of slow release 4-ASA with 1.5 g/d of slow release 5-ASA (both coated with Eudragit L and designed to dissolve at pH 6.4) as maintenance therapy in patients with quiescent Crohn's ileocolitis (Schreiber S, Howaldt S, Raedler A. Oral 4-aminosalicylic acid versus 5-aminosalicylic acid slow release tablets. Double blind, controlled pilot study in the maintenance treatment of Crohn's ileocolitis. Gut 1994 August: 35 (8):1081-5). The relapse rates after one year were virtually identical (36% and 38%, respectively). Without a placebo arm, it is unclear whether this relapse rate is materially different from the natural relapse rate, but there was no difference between the 4-ASA and the 5-ASA arms.

4-Aminosalicylic Acid in Other Conditions:

Per-oral 4-ASA was one of the three major drugs used to clear the western world of tuberculosis with doses as high as 20 g per day reported. It is bacteriostatic with an MIC under 2 μg/ml. In its initial formulation (sodium para-aminosalicylate), 4-ASA was rapidly released into the stomach and decarboxylated under acid conditions to form the toxic aminophenol with resultant gastrointestinal distress. Peak blood levels over 100 μg/ml were usual after dosing with the rapid-release 4-ASA. However, with a half-life of 50 minutes, levels were below 1 μg/ml at 8 hours and it was necessary to repeat the dose 3 times daily in order to maintain adequate anti-bacterial levels. Given the gastrointestinal side effects, compliance was poor. Another side-effect of the rapidly-absorbed 4-aminosalicylic acid preparations was drug-induced hepatitis (0.5%) usually appearing within 3 months of the start of therapy with a rash, followed by fever and less frequently by anorexia, nausea or diarrhea (see, PASER® labeling). Discontinuation resulted in recovery, but failure to recognize the reaction could result in death.

The delayed-release granular PASER® product was specifically designed to overcome the aminophenol-induced gastrointestinal distress associated with immediate release 4-ASA and to effect a more constant blood level throughout the day. In fact, with the PASER® formulation, gastrointestinal distress has effectively been eliminated and essentially no meta-aminophenol is formed. Unlike earlier 4-ASA preparations, PASER® provides 4-ASA as the free base without any sodium, calcium or sugar.

PASER® is FDA-approved for up to 12 grams per day (4 g orally Q8 h or TID) in the treatment of multi-drug resistant tuberculosis (MDRTB) and is currently used worldwide in the treatment of MDRTB, where the expected duration of treatment is for two years under direct observation on a twice or thrice daily basis. The treatment is extremely well tolerated.

Adverse Effects of 4-Aminosalicylates:

Gastro-intestinal side-effects are common with the immediate-release forms of 4-ASA and include nausea, vomiting, diarrhea and abdominal pain. The diarrhea may develop a few days into the treatment, is often self-limited, and can be relieved promptly with diphenoxylate (Peloquin C A, Henshaw T L, Huitt G A, Berning S E, Nitta A T, and James G T. Pharmacokinetic evaluation of p-aminosalicylic acid granules. Pharmcother. 1994, 14:P-2). Hypersensitivity reactions have been reported in 5% to 10% of adults, usually during the first few weeks of treatment and include fever, skin rashes, arthralgia, lymphadenopathy, hepatosplenomegaly, and rarely a syndrome resembling infections mononucleosis. There have been rare occurrences of jaundice, hepatitis, pericarditis, hypoglycemia, optic neuritis, encephalopathy, Loeffler's syndrome, and renal failure. Agranulocytosis, eosinophilia, leucopenia, and thrombocytopenia have been reported, as has Coomb's positive hemolytic anemia in patients with glucose-6-phosphate dehydrogenase deficiency (PASER® labeling).

Since the release of PASER® in 1994 and despite its widespread international use, no new problems have emerged and it appears that the side-effect profile is minimal.

Pharmacokinetics of PASER®:

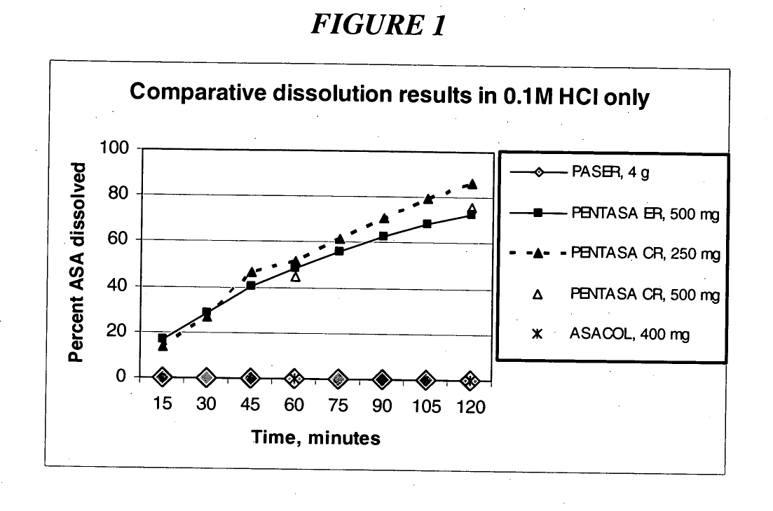

In vitro, there is no dissolution of 4-ASA from PASER® granules in simulated gastric contents even after 2 hours (see, FIG. 1). Nor is there any dissolution of 5-ASA from ASACOL 5-ASA 400 mg tablets. On the other hand, controlled-release capsules of 5-ASA (Pentasa 250 mg) and extended-release tablets of 5-ASA (Pentasa ER 500 mg) display 26.98% dissolution and 28.3% dissolution respectively at 30 minutes and 86% and 72% dissolution respectively at 2 hours, as shown is FIG. 1. The new Pentasa CR 500 mg capsule preparation behaves no differently from the ER preparation, with 78% 5-ASA dissolution by 2 hours in simulated gastric contents.

Accordingly, the present invention is directed, in part, to methods of treating inflammatory bowel disease in a patient, comprising the step of:

administering orally or by enema to said patient for a treatment period sufficient to control acute flares of said inflammatory bowel disease a solid dosage form comprising:

a therapeutically effective amount of an aminosalicylate active ingredient selected from the group consisting of 4-aminosalicylic acid, 5-aminosalicylic acid, a pharmaceutically acceptable salt thereof, or a combination thereof;

a first rate-controlling polymer that forms a pH labile enteric coating for administration of said solid dosage form orally;

optionally, a first rate-controlling polymer that forms a pH labile enteric coating for administration of said solid dosage form by enema;

wherein said dosage form releases drug at a therapeutically effective rate and in a region of the intestinal tract of said patient where said acute flares are present.

In certain embodiments, the inflammatory bowel disease is ulcerative colitis. In other embodiments, the inflammatory bowel disease is Crohn's disease.

In preferred embodiments, the active ingredient is 4-aminosalicylic acid (free acid) or the sodium, potassium, or calcium salt thereof. Preferably, the active ingredient is the free acid of 4-aminosalicylic acid.

In preferred embodiments, the solid dosage form is administered orally; and the pH labile enteric coating is present.

In preferred embodiments, the solid dosage form is administered by enema.

In certain preferred embodiments, the pH labile enteric coating begins to dissolve at about pH 5.5 to about pH 6.0. In certain other preferred embodiments, the pH labile enteric coating begins to dissolve at about pH 6.0 to about pH 6.5. In yet other preferred embodiments, the pH labile enteric coating begins to dissolve at about pH 6.5 to about pH 7.0.

In preferred embodiments where the patient is an adult, the method delivers in a 24-hour period between about 4 grams to about 12 grams of said active ingredient to the small bowel, the large bowel, or both small and large bowels of said patient.

In preferred embodiments where the patient is a child, the method delivers in a 24-hour period said therapeutically effective amount for said child is an amount proportionally adjusted based on adult dosing guidelines to the small bowel, the large bowel, or both small and large bowels of said patient.

In preferred embodiments, the dosage form releases in USP 23-NF 18:

less than about 5%, by weight, of said active ingredient over 2 hours at pH 1.0, for example, using 0.1 N HCl, which simulates gastric fluid; and

about 40%, by weight, to about 60%, by weight, of said active ingredient in about 1 hour at pH 7.2.

In preferred embodiments, the dosage form dosage form releases in USP 23-NF 18:

less than about 5%, by weight, of said active ingredient over 2 hours at pH 1.0; and

about 60%, by weight, to about 80%, by weight, of said active ingredient in about 2 hours at pH 7.2.

In preferred embodiments, the dosage form dosage form releases in USP 23-NF 18:

less than about 5%, by weight, of said active ingredient over 2 hours at pH 1.0; and

at least about 85%, by weight, of said active ingredient in about 4 hours at pH 7.2. In certain preferred embodiments, this dosage form releases in USP 23-NF 18 at least about 85%, by weight, of said active ingredient in about 4 hours at pH 6.5. In certain other preferred embodiments, this dosage form releases in USP 23-NF 18 about 25%, by weight, to about 85%, by weight, of said active ingredient in about 4 hours at pH 6.5. In yet other preferred embodiments, this dosage form releases in USP 23-NF 18 less than about 25%, by weight, of said active ingredient in about 4 hours at pH 6.5.

In preferred embodiments, the dosage form treatment period sufficient to control said acute flares does not exceed eight weeks, preferably, six weeks, more preferably, five weeks, even more preferably, four weeks.

In preferred embodiments, treatment period sufficient to control said acute flares does not exceed the time required to restore pre-flare disease status.

In preferred embodiments, the wherein said therapeutically effect amount for a child is proportionally adjusted based on adult dosing guidelines.

In preferred embodiments, the solid dosage form is in granular form.

In preferred embodiments, the solid dosage form is co-administered with an acid-containing food or beverage, preferably orange juice, grapefruit juice, apple juice, tomato juice, apple sauce, or a combination thereof. As used herein, the term “co-administered” means at same time or simultaneously (that is, together), or in any order provided that the active ingredient and the acid-containing food or beverage are taken by the patient not more than about 30 minutes apart, even more preferably more than about 15 minutes apart, and still more preferably more than about 5 minutes apart.

In preferred embodiments, the solid dosage form is the form of generally spherical particles having a diameter of about 1.3 mm to about 3.5 mm, preferably about 1.3 mm to about 3.0 mm, and more preferably, about 1.3 mm to 2.5 mm.

In preferred embodiments, the dosage form further comprising at least one second rate-controlling polymer, whose function is at least in part, to prevent release of active drug from the dosage form during gastric transit, if administered orally. The enteric coating and the at least one second rate prevent the release of the active ingredient during transit through the stomach and proximal small intestine allowing drug delivery specifically to the ileocecal region and the large intestine.

Suitable materials for the second rate-controlling polymer include, but are not limited, to hydroxyalkyl cellulose, poly(ethylene)oxide, microcrystalline cellulose, alkyl cellulose, carboxymethyl cellulose, hydrophilic cellulose derivatives, polyethylene glycol, or a combination thereof. Preferably, the hydroxyalkyl cellulose is hydroxypropyl cellulose, hydroxypropylmethyl cellulose, or a combination thereof.

In certain preferred embodiments, the at least one second rate-controlling polymer is a combination of microcrystalline cellulose and hydroxyalkyl cellulose, especially a combination of microcrystalline cellulose and hydroxypropylmethyl cellulose.

In preferred embodiments, the active ingredient is present in a range of about 50%, by weight, to about 95%, by weight, and the at least one second rate-controlling polymer is present in a range of about 5%, by weight, to about 50%, by weight, based on the total weight of said active ingredient and said at least one second rate-controlling polymer. In certain more preferred embodiments, active ingredient is present in a range of about 65%, by weight, to about 85%, by weight; and said at least one second rate-controlling polymer is present in a range of about 15%, by weight, to about 15%, by weight, based on the total weight of said active ingredient and said at least one second rate-controlling polymer.

In preferred embodiments, the pH labile enteric coating comprises an inner layer and an outer layer.

In preferred embodiments, the inner layer is formed from a composition comprising:

talc;

methacrylic acid (co)polymer; and

a polymerizing agent, such as, for example, dibutyl sebecate.

In preferred embodiments, the outer layer comprises hydroxypropylmethyl cellulose.

In preferred embodiments, the at least one second rate-controlling polymer is:

about 90%, by weight, to about 99.9%, by weight, of microcrystalline cellulose; and

about 0.1%, by weight, to about 10%, by weight, of hydroxypropylmethyl cellulose, based on the total weight of said at least one second rate-controlling polymer.

In another aspect, the invention is directed to granular dosage forms, comprising:

generally spherical particles comprising:

an active ingredient selected from the group consisting of 4-aminosalicylic acid, 5-aminosalicylic acid, a pharmaceutically acceptable salt thereof, or a combination thereof;

a first rate controlling polymer that forms a pH labile enteric coating; and

at least one second rate-controlling polymer;

wherein said particles have a diameter of about 1.3 mm to about 3.5 mm (preferably about 1.3 mm to about 3.0 mm, more preferably, about 1.3 mm to 2.5 mm); and

wherein said dosage form releases in USP 23-NF 18:

less than about 5%, by weight, of said active ingredient over 2 hours at pH 1.0; and

about 40%, by weight, to about 60%, by weight, of said active ingredient in about 1 hour at pH 7.2.

The various dosage forms are prepared in accordance with acceptable pharmaceutical procedures, such as described in Remington's Pharmaceutical Sciences, 17th edition, ed. Alfonoso R. Gennaro, Mack Publishing Company, Easton, Pa. (1985).

In preferred embodiments, the granular dosage form releases in USP 23-NF 18:

less than about 5%, by weight, of said active ingredient over 2 hours at pH 1.0; and

about 60%, by weight, to about 80%, by weight, of said active ingredient in about 2 hours at pH 7.2.

In preferred embodiments, the granular dosage form releases in USP 23-NF 18:

wherein said dosage form releases in USP 23-NF 18:

less than about 5%, by weight, of said active ingredient over 2 hours at pH 1.0; and

at least about 85%, by weight, of said active ingredient in about 4 hours at pH 7.2.

In preferred embodiments of the granular dosage form,

the active ingredient is present in a range of about 50%, by weight, to about 95%, by weight; and

the at least one second rate-controlling polymer is present in a range of about 5%, by weight, to about 50%, by weight;

based on the total weight of the active ingredient and the at least one second rate-controlling polymer.

In preferred embodiments of the granular dosage form,

the active ingredient is present in a range of about 65%, by weight, to about 85%, by weight; and

the at least one second rate-controlling polymer is present in a range of about 15%, by weight, to about 35%, by weight;

based on the total weight of the active ingredient and the at least one second rate-controlling polymer.

In preferred embodiments of the granular dosage form, the pH labile enteric coating comprises an inner layer and an outer layer.

In preferred embodiments of the granular dosage form, the inner layer is formed from a composition comprising:

talc;

methacrylic acid (co)polymer; and

a polymerizing agent, such as, for example, dibutyl sebecate.

In preferred embodiments of the granular dosage form, the outer layer comprises hydroxypropylmethyl cellulose.

In preferred embodiments of the granular dosage form, the second rate-controlling polymer is hydroxyalkyl cellulose, poly(ethylene)oxide, microcrystalline cellulose, alkyl cellulose, carboxymethyl cellulose, hydrophilic cellulose derivatives, polyethylene glycol, or a combination thereof. Preferably, hydroxyalkyl cellulose is hydroxypropyl cellulose, hydroxypropylmethyl cellulose, or a combination thereof. In certain preferred embodiments, the at least one second rate-controlling polymer is a combination of microcrystalline cellulose and hydroxyalkyl cellulose, especially a combination of microcrystalline cellulose and hydroxypropylmethyl cellulose.

In preferred embodiments of the granular dosage form, the at least one second rate controlling polymer is about 90%, by weight, to about 99.9%, by weight, of microcrystalline cellulose and about 0.1%, by weight, to about 10%, by weight, of hydroxypropylmethyl cellulose, based on the total weight of said at least one second rate-controlling polymer.

In another aspect, the invention is directed to kits, comprising:

instructions for administering an oral dosage form to a patient suffering from inflammatory bowel disease;

the granular oral dosage form described above; and

at least one oral delivery vehicle.

In yet other aspects, the invention is directed to kits, comprising:

instructions for administering an enema to a patient suffering from inflammatory bowel disease;

the granular enema dosage form described above; and

at least one enema delivery vehicle.

The compounds of this invention may be administered orally or by enema and are administered in combination with conventional pharmaceutical carriers that control release of the active ingredient to enable controlled-release of the active ingredient in the proper region of the GI tract or otherwise facilitate the manufacture of the dosage form or optimize the physical and/or pharmaceutical characteristics of the dosage form Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, methyl cellulose, alkyl cellulose, sodium carboxymethyl cellulose, polyvinylpyrrolidine, low melting waxes, and ion exchange resins.

The aminosalicylate active ingredients useful in the invention may be prepared by synthetic techniques that are well known in the art. See, for example, U.S. Pat. No. 427,564 (1890) and by Sheehan, J. Am. Chem. Soc. 70, 1665 (1948) that describe the synthesis of 4-aminosalicylic acid. 5-Aminosalicylic acid can be prepared according to Le Guyader, Peltier, Compt. Rend. 253, 2544 (1961) (see Merck Index). The conversion of the commercially available sodium salt of 4-aminosalicylic acid can be converted to the free base by aqueous acidification with hydrochloric acid.

All forms of the compounds useful in the dosage forms of the invention, including free acid, free base, and zwitterions, isomorphic crystalline forms, all chiral, enantiomeric, racemic forms, hydrates, solvates, salts and acid salt hydrates, are contemplated to be within the scope of the present invention.

Certain active ingredients useful in the methods and dosage forms of the invention may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Suitable oral dosage forms include, but are not limited to, tablets, pills, capsules, sachets, granules, powders, chewing gums, suspensions, emulsions, suppositories, and solutions. Particularly preferred for oral use are granules. Where appropriate and necessary the formulations may include diluents, binding agents, dispersing agents, surface-active agents, lubricating agents, coating materials, flavoring agents, coloring agents, sweeteners or any other pharmaceutically acceptable additives, for example, gelatin, sodium starch glycolate, lactose, starch, talc, magnesium stearate, microcrystalline cellulose, Povidone, hydrogenated or unsaturated oils, polyglycols, syrups or other aqueous solutions. Where the formulations are tablets or capsules and the like the oral dosage forms may be presented as premeasured unit doses or in multidose containers from which the appropriate unit dose may be withdrawn.

Preferably the oral dosage form is in single unit dosage form, e.g. as tablets, capsules, sachets, powders, solutions, suspensions, emulsions, packets, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids or solids. The unit dosage form can be, for example, a capsule or tablet itself, or it can be the appropriate number of any such compositions in package form.

In addition to standard pharmaceutical additives there may be included within oral dosage form other therapeutic agents.

The dosage forms of the invention are enteric-coated. By enteric coating the active ingredients, it is possible to control the release of these components in the gastrointestinal tract such that these components are not released in the stomach but rather is released in the small intestines or, alternatively, not released in the stomach or the proximal small intestines but are released in the distal ileum and the large bowel. In many embodiments, the controlled release is pH dependent. For example, in certain preferred embodiments, the pH labile enteric coating begins to dissolve at about pH 5.5 to about pH 6.0. In certain other preferred embodiments, the pH labile enteric coating begins to dissolve at about pH 6.0 to about pH 6.5. In yet other preferred embodiments, the pH labile enteric coating begins to dissolve at about pH 6.5 to about pH 7.0.

In addition, dosage forms of the present invention can be in the form of capsules wherein one active ingredient is compressed into a tablet or in the form of a plurality of microtablets, particles, granules or non-pareils, which are then enteric coated. These enteric-coated microtablets, particles, granules or non-pareils are then placed into a capsule or packet, or compressed into a capsule along with a granulation of any other components.

The present invention is further defined in the following Examples, in which all parts and percentages are by weight, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

PASER® granules, 4-aminosalicylic acid, which is commercially-available from Jacobus Pharmaceutical Company, Inc., Princeton, N.J., USA, is to be tested in randomized double-blind study in the management of patients (adults and children) experiencing an acute flare of Crohn's disease. The testing protocol follows below.

### Pharmacokinetics of PASER®:

In vitro, there was no dissolution of 4-ASA from PASER® granules in simulated gastric contents even after 2 hours, as shown in FIG. 1. Nor was there any dissolution of 5-ASA from ASACOL 5-ASA 400 mg tablets. On the other hand, controlled-release capsules of 5-ASA (Pentasa 250 mg) and extended-release tablets of 5-ASA (Pentasa ER 500 mg) display 26.98% dissolution and 28.3% dissolution, respectively, at 30 minutes and 86% and 72% dissolution respectively at 2 hours, as shown in FIG. 1. The new Pentasa CR 500 mg capsule preparation behaves no differently from the ER preparation, with 78% 5-ASA dissolution by 2 hours in simulated gastric contents.

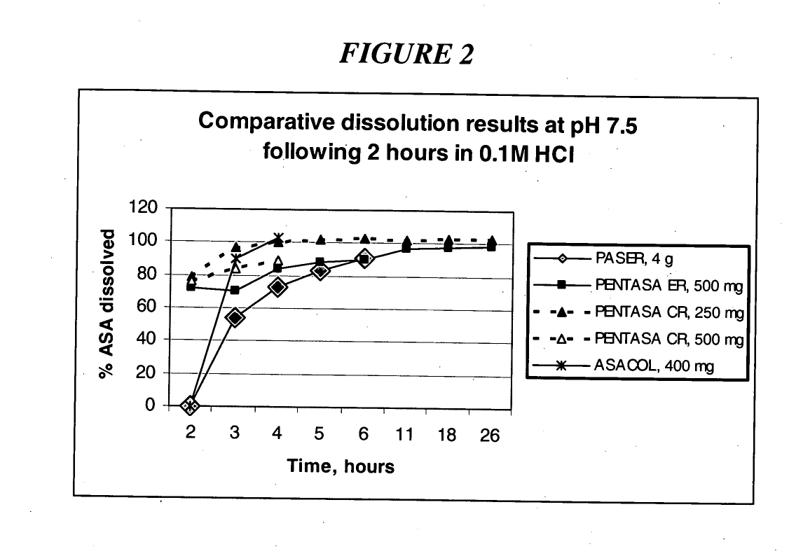

Upon transfer into pH 7.5 buffer after 2 hours in acid medium (0.1 M HCl) PASER® displayed nearly linear dissolution beginning at hour 3.5, so that 90% is dissolved after hour 6 (4 hours at pH 7.5), as shown in FIG. 2. Of the 5-ASA products, PENTASA CR 250 mg and ASACOL 400 mg were nearly completely dissolved within one hour of being transferred into pH 7.5 buffer. The 500 mg ER and CR formulations of PENTASA displayed delayed and slow release, respectively, reaching more than 80% dissolution within 2 hours post-transfer into pH 7.5 buffer.

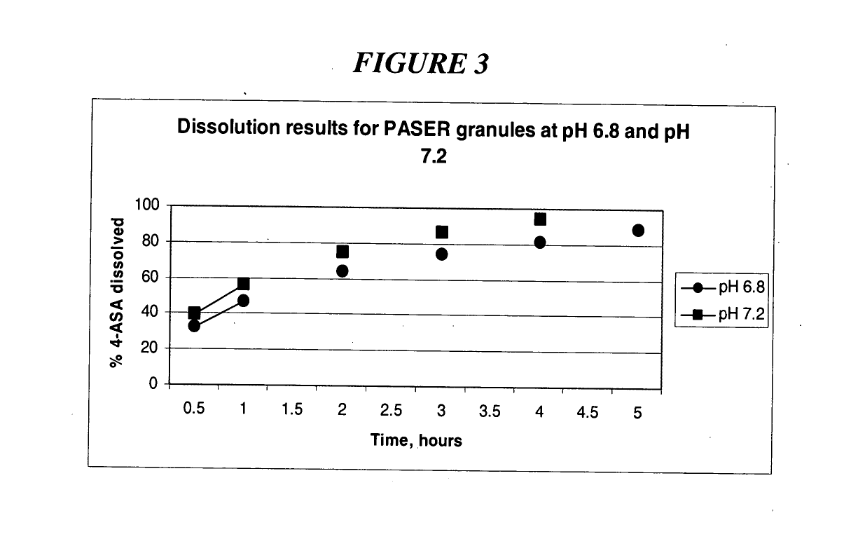

In pH 7.2 buffer, 56.4% of PASER® was dissolved after 1 hour and 94.5% was dissolved after 4 hours. The comparable numbers in pH 6.8 buffer were 47% after 1 hour and 81.6% after 4 hours, as shown in FIG. 3.

These dissolution studies support the expectation that there should be sustained and steady release of 4-ASA throughout the small intestine and likely into the proximal colon, particularly if the transit time is rapid.

0071.500 As noted above, data on dissolution at pH 5.5, 6.0 and 6.5 needs to be put into this section.

The average Tₘₐₓ for absorption/release of 4-ASA after a single oral dose of 4 grams PASER® in normal healthy volunteers is 7.95 hours (range, 1.5-24 h) with a median of 6.00 hours, Cmaxof 20.23 μg/mL ±8.8 μg/mL (range 9.36 μg/mL-35.40 μg/mL), and a half-life estimated at 1.62±0.85.¹ Four grams of 4-ASA delivered through the PASER® formulation on a once daily schedule for 8 days in 6 subjects produced a median Cmaxof 23.40 μg/mL and a median Tmax of 6.0 h on day 8, with median serum 4-ASA levels of 23.4 μg/mL, 3.7 μg/mL and 0.0 μg/mL at 4, 8 and 12 hours respectively.² Twice daily dosing in 6 subjects produced median serum concentrations of 25.8 μg/mL, 23.2 μg/mL, and 16.4 μg/mL at 4, 8, and 12 hours respectively.² Further absorption studies in 40 patients of all races showed comparable results.² Dosing PASER® at 4 grams 3 times per day (every 8 hours) in 6 patients with multi-drug resistant tuberculosis showed that the median concentration of 4-ASA after steady state had been attained (i.e: following dose number 22 on day 8) was 28.51 μg/mL (mean 28.50±14.39 μg/mL, range 1.3-56.79 μg/mL), nearly 3 times higher than the level following the initial dose (9.57 μg/mL; mean 11.01±10.14 μg/mL, range 0.37-30.16 μg/mL).¹ Pharmacokinetic parameters of 4-ASA delivered through the PASER® formulation in adults are not modified by changes in gastric pH induced by antacids or orange juice, whereas concomitant intake of high-fat food can increase Cₘₐₓ and AUC. ¹Peloquin C A, Henshaw T L, Huitt G A, Berning S E, Nitta A T, and James G T. Pharmacokinetic evaluation of p-aminosalicylic acid granules. Pharmcother. 1994, 14:P-2²Peloquin C A, Berning S E, Huitt G A, Childs J M, Singleton M D and James G T. Once-Daily and Twice-Daily Dosing of p-Aminosalicylic Acid Granules. American Journal of Respiratory Critical Care Medicine. 1999; 159:932-934

These in vivo data demonstrate that there is sustained and continuous intestinal exposure to and absorption of 4-ASA via the PASER® vehicle. Given the data on two and three times daily dosing, there may be a significant local concentration not just throughout the small intestine, but even into the proximal portion of the large intestine, depending on the individual's transit time. At 4 grams two or three times daily doses (i.e. 8 to 12 grams) of locally delivered 4-aminosalicylic acid, a therapeutic response in small bowel Crohn's disease could be expected. PASER® is already FDA-approved and utilized world-wide for the treatment of multi-drug resistant tuberculosis in doses of up to 12 grams per day (4 g orally Q8 h or TID).

Metabolism of 4-Aminosalicylic Acid:

4-ASA (or PAS, para-aminosalicylic acid) is rapidly metabolized, beginning in the gastrointestinal tract and continuing in the liver. Acetyl-PAS (Ac-PAS) can be detected in the serum before PAS appears, consistent with first-pass metabolism. Most of the 4-ASA in the PASER® formulation can be recovered in the urine, primarily as Ac-PAS. Combining the median amount excreted for PAS and Ac-PAS, 2607.30 mg of the 4000 mg dose can be recovered in the urine.³ As much as 80% of 4-ASA is excreted in the urine with 50% or more of the dosage excreted in acetylated form. The half-life of free 4-ASA in renal disease is increased from 26.4 to 30.8 minutes, and the half-life of the acetylated metabolite is increased to 309 minutes in uremic patients compared with 51 minutes in normals.⁴ ³Peloquin C A, Henshaw T L, Huitt G A, Berning S E, Nitta A T, and James G T. Pharmacokinetic evaluation of p-aminosalicylic acid granules. Pharmcother. 1994, 14:P-2⁴Held H and Fried F. Elimination of Para-Aminosalicylic Acid in Patient with Liver Disease and Renal Insufficiency. Chemotherapy 1977:23:405-415

Stability and Storage of PASER®:

Aminosalicylic acid deteriorates rapidly when exposed to moisture, heat, or light. PASER® granules should not be used if the airtight package containing the drug is swollen, indicating improper storage, or if the granules are a dark brown or purple color, indicating deterioration. Solutions or mixtures of aminosalicylic acid should be used within 24 hours and only if there has been no alteration in color.

PASER® packets should be stored at temperatures below 15° C. (59° F.), in a refrigerator or freezer, prior to dispensing. Patients should store the packets in a refrigerator or freezer but may keep them at room temperature for short periods of time. Excessive heat should be avoided.

Pharmacokinetics of Other Reported Formulations of 4-ASA:

Comparisons of PASER®, a free base formulation of 4-ASA, with assorted 4-ASA salts has demonstrated a much lower Cₘₐₓ for PASER®, a much longer Tₘₐₓ and a lower AUC_{0-∞,5} all supporting the improved clinical tolerability of the formulation and enhanced drug delivery to the affected sites in the intestinal lumen. ⁵Peloquin C A, Henshaw T L, Huitt G A, Berning S E, Nitta A T, and James G T. Pharmacokinetic evaluation of p-aminosalicylic acid granules. Pharmcother. 1994, 14:P-2.

| | Dose | Cₘₐₓ | Tₘₐₓ | AOC_{0-∞} |
|---|---|---|---|---|
| | (gm) | (μg/mL) | (h) | (μg*h/mL) |
| PASER ® | 4.0 | 49.98 | 3.54 | 209.07 |
| Na+ PAS | 2.8 | 155.44 | 0.83 | 313.22 |
| Ca++ PAS | 2.6 | 139.51 | 1.02 | 326.83 |
| K+ PAS | 2.6 | 121.09 | 1.10 | 313.22 |

Data taken from, Table 4⁵

An uncoated (hydroxypropylmethylcellulose) capsule containing 400 mg 4-ASA resulted in the immediate release of 4-ASA in the stomach in normal volunteers with plasma concentrations over time of 4-ASA and the N-acetyl metabolite closely paralleling those following the intravenous administration of 4-ASA.32 Average peak plasma concentration at 40 minutes was 16 μg/mL.

Specially coated 4-ASA capsules (amylose and ethylcellulose plasticized with dibutyl sebacate) showed great variability in time to pass through the pylorus, to reach the ileo-cecal junction and to reach the colon.⁶ In four of the 7 normal healthy volunteers, drug release was generally slow and sustained with release predominantly occurring after passage into the colon. However, in three of the volunteers, no N-acetyl-4-ASA at all was detected in the plasma. Thus 43% of normal healthy volunteers showed no evidence of absorbing any 4-ASA from this delivery system. It is doubtful that there would have been local availability and activity of 4-ASA in the colon that would have gone entirely undetected in the plasma. Plasma concentration of 4-ASA was reported for only 1 of these 7 volunteers, with detectable concentrations starting 8 hours after ingestion and maximal concentrations of 1.5 μg/mL noted at 10 and 11 hours. Levels were decreasing by 12 hours and there is no data reported beyond 12 hours. While this preparation might have some activity in the colon for some people, there is nothing to support activity in the small bowel. ⁶Tuleu C, Basirt A W, Waddington W A, Ell P J, Newton J M. Colonic delivery of 4-aminosalicylic acid using amylose-ethylcellulose-coated hydroxypropylmethylcellulose capsules. Aliment Pharmacol Ther 2002; 16:17671-1779

Pharmacokinetics of 4-ASA in Children:

Early studies of immediate-release 4-ASA (both 4-ASA administered in capsules or as a 25% suspension in water and the sodium dehydrate administered as a 10% aqueous solution) in tuberculosis established that 4-ASA pharmacokinetics in children are similar to those in adults.⁷ Dosages of 300 mg/kg/day divided into 5 equal doses (every 4 hours while awake) were studied in 4 children aged 9 months to 12 years. Serum levels of 4-ASA reached as much as 120 micrograms/ml 60 minutes after taking in an oral solution. Rapid excretion was documented with serum levels dropping to 2-2.5 mg percent after 4 hours. ⁷Söderhjelm L. Serum para-aminosalicylic acid (PAS) following oral ingestion in children. Texas Reports Biol & Med. 1949; 7:471-479.

Mechanism of Action of Aminosalicylates:

A multitude of anti-inflammatory and immunoregulatory effects have been demonstrated for aminosalicylates including inhibition of TNF-α-mediated cell proliferation and activation.⁸ Like 5-ASA, 4-ASA displays protective antioxidant activity, inhibiting lipid peroxidation and acting as a potent free radical scavenger.⁷ However, unlike 5-ASA, 4-ASA does not appear to decrease the ratio of leukotriene B4 to prostaglandin E2 when mucosal cells are exposed to the calcium ionophore A23187. And unlike 4-ASA, 5-ASA has no anti-tuberculous activity. This suggests that the mechanisms of, action of 5-ASA and 4-ASA may differ in some ways. ⁸Klotz U. The Role of Aminosalicylates at the Beginning of the New Millennium in the Treatment of Chronic Inflammatory Bowel Disease. European Journal of Clinical Pharmacology. 2000; 56:353-362

With the ability to administer as much as 12 g of topical aminosalicylates daily delivered to the diseased areas in the form of PASER® delayed-release granules, aminosalicylates can be effective in the treatment of flares of ileocecal Crohn's disease. Such treatment will improve the quality of life of Crohn's patients with flares, limit or delay the need to resort to more toxic and costly modalities and/or surgery and limit the progression of the disease over time.

Cell Turnover and Time to Response:

Cell turnover rates for the small intestine, the distal colon and the rectal mucosa are all approximately 1 to 2 days.⁹ Topical aminosalicylates (4-ASA and 5-ASA) act rapidly in rectal and colonic tissues affected by ulcerative colitis with significant clinical improvement (79%), sigmoidoscopic improvement (78%), and histologic improvement (44%) documented within 2 weeks.¹⁰ This rapid improvement indicates that significant healing can occur within a period of just a few cell cycles. ⁹Lipkin M, Sherlock P, Bell B. Cell Proliferation Kinetics in the Gastrointestinal Tract of Man. II. Cell Renewal in Stomach, Ileum, Colon, and Rectum. Journal of Clinical Investigation¹⁰Campieri A, Lanfranchi G A, Bertoni F, Brignola C, Bazzocchi G, Minguzzi M R, and Labo G. A Double-Blind Clinical Trial to Compare the Effects of 4-Aminosalicylic Acid to 5-Aminosalicylic Acid in Topical Treatment of Ulcerative Colitis. Digestion. 1984; 29:204-208

### Measures of Activity of Crohn's Disease

Crohn's Disease Activity Index:

With no gold-standard for assessing clinical changes in Crohn's disease, the Crohn's Disease Activity Index (CDAI) is the generally accepted proxy.^{11 12 13 14 15} The CDAI score has been the subject of much discussion. The baseline CDAI in all prospective trials must rely on patient recall of the events of the preceding week, although the CDAI was developed from a prospective daily diary. Efforts to validate the retrospective methodology for the baseline assessment have been limited.¹⁶ It is of note that the body weight variable, originally removed in the statistical model, was reinserted because of “the importance intuitively attributed to it by the participating physicians”.¹⁷ Other variables that seem intuitively important that were dropped in the derivation of the index include “bowel movements which awaken patients from sleep” and the “number of days per week with blood in the stool”. We will record both of those parameters and assess them in the patient's daily diary and as part of an augmented CDAI index, recognizing that blood in the stool is very infrequent in ileocecal Crohn's disease. ¹¹Best W R, Becktel J M and Singleton J W. Rederived Values of the Eight Coefficients of Crohn's Disease Activity Index (CDAI). Gastroenterology. 1979; 77:843-846¹²Harvey R F, and Bradshaw J M. A simple Index of Crohn's Disease Activity. The Lancet. Mar. 8, 1980; Page 514.¹³Van Hees P, Van Elteren P H, Van Lier H J J and Van Tongeren J H M. An Index of Inflammatory Activity in Patients with Crohn's Disease. Gut. 1980. 21:279-286.¹⁴DeDombal F T and Softley A. IOIBD* Report No 1: Observer Variation in Calculating Indices of Severity and Activity in Crohn's Disease (Special Report). Gut. 1987; 28:474-481¹⁵Brignola C, Campieri M, Bazzocchi G, Farruggia P, Tragnone A, and Lanfranchi G A. A Laboratory Index for Predicting Relapse in Asymptomatic Patients with Crohn's Disease. Gastroenterology. 1986; 91:1490-1494¹⁶Frenz M B, Dunckley P, Camporota L, Jewell D P, Travis S P L. Comparison between Prospective and Retrospective Evaluation of Crohn's Disease Activity Index Am J Gastroenterol 2005; 100:1117-1120¹⁷Best W R, Becktel J M, Singleton J W and Kern F. Development of A Crohn's Disease Activity Index. National Cooperative Crohn's Disease Study. Gastroenterology. 1976; 70 (3):439-444.

Harvey-Bradshaw Index:

The Harvey-Bradshaw Index (HBI) is another well-validated measure of clinical disease activity with five variables rather than 8 variables as in the CDAI. Its variables “general well-being”, “abdominal pain” and “number of liquid stools per day” are based solely on the patient's recollection of the day preceding the visit. Variables “abdominal mass” and “complications” are presumably assessed by the physician and/or nurse at the time of the visit, and patients can be trained to self-report these items. This affords a measure that could be used to assess time to change from the patient's diary. Despite the differences in methodology, the correlation of the HBI with the CDAI is high (r=0.93).¹⁸ ¹⁸Harvey R F, and Bradshaw J M. A simple Index of Crohn's Disease Activity. The Lancet. Mar. 8, 1980; Page 514.

Inflammatory Bowel Disease Questionnaire

Quality of life items can be elicited in another validated measure of health-related quality of life (QOL), the Inflammatory Bowel Disease Questionnaire (IBDQ). This index can be used to assess ability of the intervention to impact quality of life. Response is defined as a change of 16 points and normal QOL consistent with clinical remission is defined as an absolute score of 170 points.¹⁹ ¹⁹Sandborn W J, Feagan B G, Hanauer S B, Lochs H, Lofberg R, Modigliani R, Present D H, Rutgeerts P, Scholmerich J, Stange E G and Sutherland L R. A Review of Activity Indices and Efficacy Endpoints for Clinical Trials of Medical Therapy in Adults with Crohn's Disease. Gastroenterology. 2002; 122:512-530.

Laboratory Measures of Crohn's Disease Activity

A variety of laboratory measurements have been assessed as measures of Crohn's disease activity.²⁰ The most commonly utilized markers are (1) hemoglobin—which is one of the variables contained in the CDAI, (2) the erythrocyte sedimentation rate (ESR) and (3) C-reactive protein (CRP). Fecal calprotectin is emerging as a measure of intestinal inflammation in Crohn's disease.^{21 22} Any of these inflammatory markers may improve within 2 weeks if treatment is effective.²³ ²⁰Andre C, Descos L, Landais P and Fermanian J. Assessment of appropriate laboratory measurements to supplement the Crohn's disease activity index. Gut 1981; 22:571-574.²¹Tibble J, Teahon K, Thjodleifsson B, Roseth A, Sigthorsson G, Bridger S, Foster R, Sherwood R, Fagerhol M, Bjarnason I. A simple method for assessing intestinal inflammation in Crohn's disease. Gut 2000; 47:506-513.²²Poullis A, Foster R, Mendall M, Fagerhol M K. Emerging role of calprotectin in gastroenterology. J Gastroenterology and Hepatology 2003; 18:756-762.²³Ito H, Takazoe M, Fukuda Y, Hibi T, Kusugami K, Andoh A, Matsumoto T, Yamamura T, Azuma J, Nishimoto N, Yoshizaki K, Shimoyama T, and Kishimoto T. A Pilot Randomized Trial of Human Anti-Interleukin-6-Receptor Monoclonal Antibody in Active Crohn's Disease. Gastroenterology 2004; 126:989-996.

### Placebo and Treatment Response Rates

Rapid clinical response has been reported with infliximab,^{24 25} with significant improvement in the CDAI and IBDQ scores two weeks after commencing weekly treatments.²⁶ By two weeks, 61% of infliximab-treated patients had responded (decrease in CDAI≧70 points) and 27% were in remission (CDAI<150 and IBDQ>170-190), compared with a response rate of 17% and a remission rate of 4% in the placebo-treated group. At 4 weeks, the placebo response and remission rates had not changed, whereas there were continued gains in the infliximab-treated groups: response rate of 65% and a remission rate of 33%.²⁷ Anti-interleukin-12²⁸ and granulocyte-macrophage stimulating factor²⁹ also appear to demonstrate significant clinical activity by 22 days and 15 days, respectively. The placebo response rate (≧100-point decrease in CDAI) at 2 weeks was nil in the anti-interleukin-12 study at day 15³⁰ but in the granulocyte-macrophage stimulating factor study, the placebo response rate was approximately 12% with a concomitant placebo remission rate (CDAI≦150) of approximately 15%.³¹ ²⁴Present D H, Rutgeerts P, Targan S, Hanauer S B, Mayer L, van Hogezand R A, Podolsky D K, Sands B E, Braakman T, Dewoody K L, Schaibble T F, and van Deventer S J H. Infliximab for the Treatment of Fistulas in Patients with Crohn's Disease. The New England Journal of Medicine. 1999; 340 (18):1398-1405.²⁵Rutgeerts P, Feagan B G, Lichtenstein G R, Mayer L F, Schreiber S, Colombel J F, Rachmilewitz D, Wolf D C, Olson A, Bao W, Hanauer S B. Comparison of Scheduled and Episodic Treatment Strategies of Infliximab in Crohn's Disease. Gastroenterology 2004; 126 (2):402-413.²⁶Targan S R, Hanauer S B, Van Deventer S J H, Mayer L, Present D H, Braakman T, DeWoody K, Schable T F, and Rutgeerts P J. A Short-Term Study of Chimeric Monoclonal Antibody cA2 to Tumor Necrosis Factor α for Crohn's Disease. The New England Journal of Medicine. 1997; 337 (15):1029-1035.²⁷Targan S R, Hanauer S B, Van Deventer S J H, Mayer L, Present D H, Braakman T, DeWoody K, Schable T F, and Rutgeerts P J. A Short-Term Study of Chimeric Monoclonal Antibody cA2 to Tumor Necrosis Factor α for Crohn's Disease. The New England Journal of Medicine. 1997; 337 (15):1029-1035.²⁸Mannon P J, Fuss I J, Mayer L, et. al. Anti-Interleukin-12 Antibody for Active Crohn's Disease. New Eng J Med 2004; 351:2069-79.²⁹Krozenik J R, B K Dieckgraefe, J F Valentine, D F Hausman, M J Gilbert. Sargramostim for Active Crohn's Disease. N Engl J Med 2005; 352:2193-1201.³⁰Mannon P J, Fuss I J, Mayer L, et. al. Anti-Interleukin-12 Antibody for Active Crohn's Disease. New Eng J Med 2004; 351:2069-79.³¹Krozenik J R, B K Dieckgraefe, J F Valentine, D F Hausman, M J Gilbert. Sargramostim for Active Crohn's Disease. N Engl J Med 2005; 352:2193-1201.

### Study Protocol:

This is a four week randomized triple-blind placebo controlled trial involving 54 patients which is designed to assess the efficacy of PASER®, a delayed-release granular formulation of 4-aminosalicylic acid to abort acute flares in patients with ileocecal Crohn's disease. The proposed study will have 2 arms, each with approximately 27 patients dosed three times per day for 2 weeks followed by 2 weeks of a twice daily regimen:
(1) PASER® 12 grams per day (4 grams three times daily) for 2 weeks followed by PASER® 8 grams per day (4 grams two times daily), and
(2) Placebo administered as a granular preparation that appears identical to the active granular preparation three times daily for 2 weeks followed by twice daily dosing for 2 weeks.

### Eligibility Criteria:

Eligible patients will be age 18 to 65 with known Crohn's disease involving the ileum and/or cecum (Vienna Classification B1L3)³² having an acute flare as defined by their physician. Harvey Bradshaw Index should be at least 7. CDAI for the week preceding entry will be assessed at the time of admission to the study using patient recall of the 3 days prior to entry along with physician examination and laboratory data. Admission will not be contingent upon the CDAI calculation so as not to cause delays. This baseline CDAI however will be the basis for determining therapeutic effect. With HBI≧7, we expect virtually all patients to have a CDAI>200 and most to have CDAI>220. ³²Gaschec, Scholmerick J, Brynskov J, et. al. A simple classification of Crohn's Disease: report of the Working Party for the World Congresses of Gastroenterology, Vienna, 1998. Inflamm Bowel Dis 2000; 6:11.

The onset of the acute flare should have been abrupt, declaring itself over 72 hours, and should have started no more than 4 weeks before study entry. Symptoms relating to the flare should not have diminished or started to improve prior to entry.

The diagnosis of Crohn's disease must have previously been established by radiography, endoscopy and/or biopsy (at least 2 of the 3 modalities) with at least one confirmatory test having been performed no more than 36 months before entry. The diagnosis must have been confirmed by at least one gastroenterologist.

Patients must give written informed consent for participation in the trial.

### Permissible Concomitant Therapy:

Patients may be using certain conventional medications, including 5-aminosalicylates by any route in stable doses prior to study entry. It is our opinion that patients who are experiencing a flare while on mesalamine derivatives or sulfasalazine are “breaking through” or “failing” 5-ASA, and not getting any measurable benefit. There is however some disagreement among consultants as to whether discontinuing 5-ASA products prior to entry would be acceptable to an ethics board. It is our position that 5-ASA should be discontinued if it is not providing any benefit and the patient has “broken through while taking 5-ASA. Given the short half-life, no wash-out period will be required.

Stable doses over the preceding 4 weeks in patients who have been maintained for at least 4 months on the immunosuppressives azathioprine (AZA), 6-mercaptopurine (6 MP) or methotrexate are permissible, as are probiotics and/or antibiotics in stable doses for 4 weeks. For those patients on AZA or 6 MP, thiopurine methyltransferase (TPMT) levels and genotyping will not be required prior to entry into the trial, as AZA/6 MP associated myelosuppression is usually not related to TPMT levels.³³ Furthermore, preliminary evidence suggests that any effect of aminosalicylates on TPMT is nor modified by treatment with AZA and is independent of TPMT phenotype.³⁴ However, as an extra precaution, all patients will have blood tests at the 2 week mark in order to ensure early recognition of an adverse hematologic effect. ³³Colombel J F, Ferrari N, Debuysere H, Marteau P, Gendre J P, Bonaz B, Soule J C, Modigliani R, Touze Y, Catala P, Libersa C, Broly F. Genotypic analysis of thiopurine S-methyltransferase in patients with Crohn's disease and severe myelosuppression during azathioprine therapy. Gastroenterology 2000; 188 (6):1025-1030.³⁴Xin H, Fischer C, Schwab M, Klotz U. Effects of aminosalicylates on TPMT activity: an ex vivo study in patients with inflammatory bowel disease. Alimentary Pharmacology and Therapeutics 2005; 21 (9):1105.

Patients requiring anticoagulants for comorbid conditions will not be excluded but may require an adjustment of dosage.

### Exclusion Criteria:

Patients receiving concomitant corticosteroids, including budesonide, will be excluded as will patients who have used corticosteroids within the previous 2 months. PASER® is expected to permit avoidance of corticosteroids and there is concern that concomitant corticosteroids may obscure the effect of aminosalicylates and potentially interfere with the activity of aminosalicylates (Physicians' Desk Reference 59 Edition 2005, p 3168. Rowasa® labeling—Solvay). Should PASER® prove effective in patients not on steroids, it will be of interest to compare PASER® to steroids and to separately examine effectiveness of PASER® in patients on steroids.

Other exclusions include patients on cyclosporine or mycophenolate mofetil or who have been treated with experimental drugs during the last three months. Patients who are on maintenance infliximab or have received infliximab less than 3 months earlier will also be excluded. Use of other biologics in the preceding 3 months will also be cause for exclusion.

Additional exclusions include failure to sign an informed consent, a diagnosis of primary sclerosing cholangitis, infectious diarrhea, signs of intestinal obstruction, perforation, new fistulization as part of the acute flare or increased activity in (a) chronic fistula(e) as part of the acute flare. Patients with allergy or sensitivity to salicylates will be excluded as will patients with severe renal disease and hepatic disease.

If the severity of the flare has already started to decrease spontaneously prior to entry, the patient will not be eligible for entry.

Pregnant women and women who are breast-feeding will be excluded, although there are no data implicating PASER® as a teratogen. In patients with tuberculosis 4-ASA has been used in all three trimesters of pregnancy. There are no data to suggest that the treatment is deleterious either to the mother or the fetus. Furthermore, PASER® is approved for use in children at correspondingly lower doses. The approved labeling for tuberculosis says that the medication should be used in pregnant patients only if really needed. Women of child-bearing age will need to agree to use adequate contraception for the 4 week period of the trial if they are sexually active.

### Duration of Study:

Given the expectation that there should be a rapid response to effective treatment in patients with ileocecal Crohn's disease, the duration of the double-blind placebo-controlled trial will be 4 weeks after entry.

### Statistics:

Estimation of Sample Size:

Assuming a placebo response (decrease in CDAI≧70 points) rate of 20% at 4 weeks and an active treatment response rate of 60% at 4 weeks,³⁵ each arm of the trial should have 27 patients in order to achieve a power of 80% with an alpha of 0.05. Assuming a placebo remission rate (decrease in CDAI≧100 points and CDAI≦150) of 5% at four weeks as opposed to 40% in the active treatment group, each arm of the trial should have 27 patients in order to achieve a power of 80% with an alpha of 0.05.³⁶ ³⁵Targan S R, Hanauer S B, Van Deventer S J H, Mayer L, Present D H, Braakman T, DeWoody K, Schable T F, and Rutgeerts P J. A Short-Term Study of Chimeric Monoclonal Antibody cA2 to Tumor Necrosis Factor α for Crohn's Disease. The New England Journal of Medicine. 1997; 337 (15):1029-1035.³⁶Fleiss J L, Levin B, Paik M C. Statistical Methods for Rates and Proportions, Third Edition. John Wiley & Sons, Inc. 2003.

Randomization and Blinding:

Patients will be randomized in predetermined blocks. The patient, the physician, and the research pharmacy will all be blind as to the specific treatment being administered. At the end of the study (i.e. at the 4 week visit) both the patient and the physician will independently report whether they believe that the patient received the active agent or the placebo. If the blinding has been effective, the responses should be entirely random, although it is noted that failure to improve may make participants suspicious that they were receiving placebo. Furthermore, there may be a higher rate of withdrawals among the patients on the placebo arm.

### Baseline Assessment:

The baseline assessment will include a detailed history pertaining to the patient's Crohn's disease and comorbid conditions, a physical examination, including weight [with shoes off and dressed] and oral temperature. CDAI (using patient recall of the 3 days prior to entry along with physician examination and laboratory data), Harvey Bradshaw Index, as well as patient and physician global assessments will be recorded. IBDQ may be assessed. A blood sample will be drawn to measure CBC with platelet count, sedimentation rate, and C-reactive protein.³⁷ Comprehensive biochemistries will include liver functions and renal parameters. A specimen will be stored for potential future examination of TPMT, 6TG levels, or other related studies. Stool samples for calprotectin and C. diff will also be collected.³⁸ ³⁷Beck I T. Laboratory Assessment of Inflammatory Bowel Disease. Digestive Diseases and Sciences. 1987; 32 (12):26S-41S.³⁸Sands B. From Symptom to Diagnosis: Clinical Distinctions Among Various Forms of Intestinal Inflammation. Gastroenterology 2004; 126:1519-1532.

### Patient Follow-Up:

The patient will be expected to maintain a diary and to return at 2 weeks for blood and stool tests (CBC, comprehensive biochemistries, specimen for potential future examination for TPMT, 6TG, 4-ASA and metabolite levels, or other related studies, ESR, CRP, calprotectin). The 2 week CBC results will be processed locally and the treating physician will be responsible for reviewing the results. The other specimens may be processed after the clinical trial has been completed and the results will not be available for clinical decision-making during the study. There will be no clinical evaluation at the 2 week mark for the study.

For patients on AZA/6 MP, should there be a significant change in hematologic parameters at 2 weeks (decrease in WBC by 1000/mm³ or to <2000/mm³, decrease in Hgb by 1 g/dl or to <8.0 g/dl, and/or decrease in platelet count by 50,000/mm³ or to <50,000/mm³), the physician will effect at least a 25% dose reduction of AZA/6 MP and report this immediately.

The patient will return 4 weeks after starting the trial for a clinical evaluation including history, collection of the diary, inquiries about side effects, and a directed physical exam including weight [with shoes off and dressed] and oral temperature. At the time of this visit, the patient will provide a global assessment of disease activity and change in disease activity. The physician will provide a global assessment of disease severity and change in severity. CDAI and Harvey-Bradshaw will be formally assessed and IBDQ may be examined. A blood sample will be drawn to measure CBC with platelet count, sedimentation rate, C-reactive protein, comprehensive biochemistries. Blood may also be tested for 4-ASA and metabolite levels, for TPMT, 6TG, or other related studies. A stool sample for calprotectin will also be collected. Compliance will be assessed by counting the remaining packets of medication.

Patients with disease progression are to be managed as their physician deems necessary. If desired, the test medication may be continued even if the patient is started on additional medications, recognizing that polypharmacy in Crohn's disease may be associated with more severe Crohn's and worsening disease status.³⁹ Introduction of cortiocosteroids, infliximab, 6-mercaptopurine, azathioprine or cyclosporine will be counted as a treatment failure. Increases in doses of permissible concomitant therapies during the one month study will be counted as a failure. No other investigational medications can be administered concurrently. ³⁹Cross R K, Wilson K T, Binion D G. Polypharmacy and Crohn's Disease. Aliment Pharmacol Ther 2005; 21:1211-1216.

Patients may drop out at any time and those with significant adverse effects requiring discontinuation of the study medication will be considered treatment failures.

### Data Analysis:

The primary analysis will be performed according to an intention-to-treat (ITT) approach, including all patients screened and randomly assigned to treatment. Statistical summaries will include the mean, median, standard deviation, minimum and maximum for quantitative variables and the number and percent of subjects for each outcome for qualitative variables. Statistical significance will be declared if the two-sided p-value is <0.05. All computations will be performed using SAS®.

### Statistical Considerations:

Baseline Demographic and Medical History:

For quantitative variables statistical treatment group comparisons will be made using a two sample t-test or a Wilcoxon Rank sum test, as appropriate. Fisher's exact test will be used to compare the treatment groups with respect to qualitative variables.

Primary End Point:

The primary study end-point will be the rate of therapeutic response as defined by:

(1) A reduction of the CDAI score of ≧70 points by 4 weeks compared with baseline, and

The treatment groups will be compared using Fisher's exact test.

If the patient experiences therapeutic response and/or remission early in the study, the response/remission must hold until completion of the four week study in order for the treatment to be considered a success. Patients who withdraw early because of treatment success will have the last observation carried forward if telephone and/or written follow-up can confirm that the treatment success has held until what would have been the 4 week mark. Patients who withdraw early for whom no follow-up can be obtained will be declared treatment failures even if the reason for withdrawal was “success.”

Secondary End Points:

The following secondary endpoints will be examined:
a. Rate of remission as defined by the decrease in CDAI≧100 points and total CDAI≦150
b. Rate of response as defined by a reduction in HBI to less than 5 by 4 weeks
c. Rate of remission as defined by the decrease in HBI to less than 3
d. Time to response and/or remission including time to change in HBI, according to elements of the daily patient diary
e. Increase in IBDQ to greater than 170 and the time to score above 170
f. The change from baseline in the patient's general sense of disease activity as recorded in the individual daily diary. The diary is intended to be completed by the patient and has been designed to avoid confusion. The scales for the patient rating range from normal to severe. These are symptoms that carry the notation in the diary of zero for normal—i.e. no symptoms to the highest number used for that component to indicate severity. While such a rating scale facilitates accurate recording for the normal state if one is to calculate a percentage response zero in the numerator or denominator is cumbersome. In the analysis we plan to award points from 1 to 4 for each of the scores selected by the patient. The overall disease activity index includes: normal (1), mild (3), moderate (5), and severe (7).⁴⁰ Change over time will be rated as much better (1), slightly better (2), the same (3), slightly worse (4), and much worse (5).
g. Absence of night time stools, if they were present on entry, and time to disappearance.
h. Time to normalization of all other components in the diary.
i. Change in Hgb, ESR, CRP, platelet count, calprotectin from baseline and time to normalization.
j. Change in global physician assessment of disease activity from baseline to study completion.⁴⁰ For patients who withdraw early, the final assessment prior to withdrawal will be used. For those patients who attained the primary endpoints, time to change will be examined. ⁴⁰Best W R, Becktel J M, Singleton J W and Kern F. Development of A Crohn's Disease Activity Index. National Cooperative Crohn's Disease Study. Gastroenterology. 1976; 70 (3):439-444
k 4-ASA and Ac-PAS levels may be assessed. TPMT and 6TG levels may be assessed.

If the patient experiences improvement and/or remission early in the study, the improvement/remission must hold until completion of the four week study in order for the treatment to be considered a success. If the patient withdraws early because of response or remission, the last assessment prior to withdrawal will be used and carried forward in the analysis only if telephone and/or written confirmation of the continued success at 4 weeks are obtained.

Agreement between patient and physician assessments will be examined.

The life table analyses will be based on the time to normal provided that the patient stays normal for the rest of the study and has reached normal 4 or more days prior to the end of the study.

The secondary variables in this study include both categorical and numerical parameters. The numerical parameters such as the physician global assessment of disease activity will be analyzed both as a change from baseline to the end of the study and the treatment groups will be compared using a two sample t-test or a Wilcoxon Rank Sum test, as appropriate. For the categorical variables, such as the absence of blood in the stool at study completion, the treatment groups will be compared using Fisher's exact test. For time-related variables, such as the time to diary normalization, a life table analysis will also be conducted. For each treatment group a Kaplan-Meier curve will be constructed. The treatment groups will be compared using a Log Rank test and a Wilcoxon test. The life table analyses will be based on the time to event (normalization of the characteristic) provided that the patient stays normal for the rest of the study and has reached normal 4 or more days prior to the end of the study.

### Safety Endpoints:

Safety will be assessed through the recording of adverse events. These events will be coded using a standard coding dictionary. For each treatment group, the number and percentage of subjects reporting an adverse event within each preferred term will be summarized. Summaries will also be developed based on severity and relationship to study drug. All serious adverse drug events will be described in detail.

When ranges are used herein for physical properties, such as molecular weight, or chemical properties, such as chemical formulae, all combinations and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating inflammatory bowel disease in a patient, comprising the step of:
administering orally or by enema to said patient for a treatment period sufficient to control acute flares of said inflammatory bowel disease a solid dosage form comprising:
a therapeutically effective amount of an aminosalicylate active ingredient selected from the group consisting of 4-aminosalicylic acid, 5-aminosalicylic acid, a pharmaceutically acceptable salt thereof, or a combination thereof;
a first rate-controlling polymer that forms a pH labile enteric coating for administration of said solid dosage form orally;
optionally, a first rate-controlling polymer that forms a pH labile enteric coating for administration of said solid dosage form by enema;
wherein said dosage form releases drug at a therapeutically effective rate and in a region of the intestinal tract of said patient where said acute flares are present.

2. A method of claim 1,
wherein said inflammatory bowel disease is ulcerative colitis.

3. A method of claim 1,
wherein said inflammatory bowel disease is Crohn's disease.

4. A method of claim 1,
wherein said active ingredient is 4-aminosalicylic acid (free acid) or the sodium, potassium, or calcium salt thereof.

5. A method of claim 1,
wherein said active ingredient is 4-aminosalicylic acid (free acid).

6. A method of claim 1,
wherein said solid dosage form is administered orally; and
wherein said pH labile enteric coating is present.

7. A method of claim 1,
wherein said solid dosage form is administered by enema.

8. A method of claim 1,
wherein said patient is an adult; and
wherein said method delivers in a 24-hour period between about 4 grams to about 12 grams of said active ingredient to the small bowel, the large bowel, or both small and large bowels of said patient.

9. A method of claim 8,
wherein said patient is a child; and
wherein said method delivers in a 24-hour period said therapeutically effective amount for said child is an amount proportionally adjusted based on adult dosing guidelines to the small bowel, the large bowel, or both small and large bowels of said patient.

10. A method of claim 1,
wherein said pH labile enteric coating begins to dissolve at about pH 5.5 to about pH 6.0.

11. A method of claim 1,
wherein said pH labile enteric coating begins to dissolve at about pH 6.0 to about pH 6.5.

12. A method of claim 1,
wherein said pH labile enteric coating begins to dissolve at about pH 6.5 to about pH 7.0.

13. A method of claim 1,
wherein said dosage form releases in USP 23-NF 18:
less than about 5%, by weight, of said active ingredient over 2 hours at pH 1.0; and
about 40%, by weight, to about 60%, by weight, of said active ingredient in about 1 hour at pH 7.2.

14. A method of claim 1,
wherein said dosage form releases in USP 23-NF 18:
less than about 5%, by weight, of said active ingredient over 2 hours at pH 1.0; and
about 60%, by weight, to about 80%, by weight, of said active ingredient in about 2 hours at pH 7.2.

15. A method of claim 1;
wherein said dosage form releases in USP 23-NF 18:
less than about 5%, by weight, of said active ingredient over 2 hours at pH 1.0; and
at least about 85%, by weight, of said active ingredient in about 4 hours at pH 7.2.

16. A method of claim 15,
wherein said dosage form releases in USP 23-NF 18:
at least about 85%, by weight, of said active ingredient in about 4 hours at pH 6.5.

17. A method of claim 15,
wherein said dosage form releases in USP 23-NF 18:
about 25%, by weight, to about 85%, by weight, of said active ingredient in about 4 hours at pH 6.5.

18. A method of claim 15,
wherein said dosage form releases in USP 23-NF 18:
less than about 25%, by weight, of said active ingredient in about 4 hours at pH 6.5.

19. A method of claim 1,
wherein said treatment period sufficient to control said acute flares does not exceed eight weeks.

20. A method of claim 1,
wherein said treatment period sufficient to control said acute flares does not exceed six weeks.

21. A method of claim 1,
wherein said treatment period sufficient to control said acute flares does not exceed five weeks.

22. A method of claim 1,
wherein said treatment period sufficient to control said acute flares does not exceed four weeks.

23. A method of claim 1,
wherein said treatment period sufficient to control said acute flares does not exceed the time required to restore pre-flare disease status.

24. A method according to claim 1,

25. A method according to claim 1,
wherein said solid dosage form is in granular form.

26. A method according to claim 1,
wherein said solid dosage form is co-administered with an acid-containing food or beverage.

27. A method according to claim 1,
wherein said solid dosage form is the form of generally spherical particles having a diameter of about 1.3 mm to about 3.5 mm.

28. A method according to claim 1,
wherein said dosage form further comprising at least one second rate-controlling polymer.

29. A method according to claim 28,
wherein said second rate-controlling polymer is hydroxyalkyl cellulose, poly(ethylene)oxide, microcrystalline cellulose, alkyl cellulose, carboxymethyl cellulose, hydrophilic cellulose derivatives, polyethylene glycol, or a combination thereof.

30. A method according to claim 28,
wherein said hydroxyalkyl cellulose is hydroxypropyl cellulose, hydroxypropylmethyl cellulose, or a combination thereof.

31. A method according to claim 1,
wherein said at least one second rate-controlling polymer is a combination of microcrystalline cellulose and hydroxyalkyl cellulose.

32. A method according to claim 1,
wherein said at least one second rate-controlling polymer is a combination of microcrystalline cellulose and hydroxypropylmethyl cellulose.

33. A method of claim 28,
wherein:
said active ingredient is present in a range of about 50%, by weight, to about 95%, by weight; and
said
said at least one second rate-controlling polymer is present in a range of about 5%, by weight, to about 50%, by weight;
based on the total weight of said active ingredient and said at least one second rate-controlling polymer.

34. A method of claim 28,
wherein:
said active ingredient is present in a range of about 65%, by weight, to about 85%, by weight; and
said at least one second rate-controlling polymer is present in a range of about 15%, by weight, to about 35%, by weight;
based on the total weight of said active ingredient and said at least one second rate-controlling polymer.

35. A method according to claim 1,
wherein said pH labile enteric coating comprises an inner layer and an outer layer.

36. A method according to claim 35,
wherein said inner layer is formed from a composition comprising:
talc;
methacrylic acid (co)polymer; and
a polymerizing agent.

37. A method according to claim 35,
wherein said outer layer comprises hydroxypropylmethyl cellulose.

38. A method according to claim 28,
wherein said at least one second rate-controlling polymer is:
about 90%, by weight, to about 99.9%, by weight, of microcrystalline cellulose; and
about 0.1%, by weight, to about 10%, by weight, of hydroxypropylmethyl cellulose,
based on the total weight of said at least one second rate-controlling polymer.

39. A granular dosage form, comprising:
generally spherical particles comprising:
an active ingredient selected from the group consisting of 4-aminosalicylic acid, 5-aminosalicylic acid, a pharmaceutically acceptable salt thereof, or a combination thereof;
a first rate controlling polymer that forms a pH labile enteric coating; and
at least one second rate-controlling polymer;
wherein said particles have a diameter of about 1.3 mm to about 3.5 mm; and
wherein said dosage form releases in USP 23-NF 18:
less than about 5%, by weight, of said active ingredient over 2 hours at pH 1.0; and
about 40%, by weight, to about 60%, by weight, of said active ingredient in about 1 hour at pH 7.2.

40. A granular dosage form of claim 39,
wherein said dosage form releases in USP 23-NF 18:
less than about 5%, by weight, of said active ingredient over 2 hours at pH 1.0; and
about 60%, by weight, to about 80%, by weight, of said active ingredient in about 2 hours at pH 7.2.

41. A granular dosage form of claim 39,
wherein said dosage form releases in USP 23-NF 18:
less than about 5%, by weight, of said active ingredient over 2 hours at pH 1.0; and
at least about 85%, by weight, of said active ingredient in about 4 hours at pH 7.2.

42. A granular dosage form of claim 39,
wherein:
said active ingredient is present in a range of about 50%, by weight, to about 95%, by weight; and
said
said at least one second rate-controlling polymer is present in a range of about 5%, by weight, to about 50%, by weight;
based on the total weight of said active ingredient and said at least one second rate-controlling polymer.

43. A granular dosage form of claim 39,
wherein:
said active ingredient is present in a range of about 65%, by weight, to about 85%, by weight; and
said at least one second rate-controlling polymer is present in a range of about 15%, by weight, to about 35%, by weight;
based on the total weight of said active ingredient and said at least one second rate-controlling polymer.

44. A granular dosage form of claim 39,
wherein said pH labile enteric coating comprises an inner layer and an outer layer.

45. A granular dosage form of claim 44,
wherein said inner layer is formed from a composition comprising:
talc;
methacrylic acid (co)polymer; and
a polymerizing agent.

46. A granular dosage form of claim 44,
wherein said outer layer comprises hydroxypropylmethyl cellulose.

47. A granular dosage form of claim 39,
wherein said second rate-controlling polymer is hydroxyalkyl cellulose, poly(ethylene)oxide, microcrystalline cellulose, alkyl cellulose, carboxymethyl cellulose, hydrophilic cellulose derivatives, polyethylene glycol, or a combination thereof.

48. A granular dosage form of claim 47,
wherein said hydroxyalkyl cellulose is hydroxypropyl cellulose, hydroxypropylmethyl cellulose, or a combination thereof.

49. A granular dosage form of claim 39,
wherein said at least one second rate-controlling polymer is a combination of microcrystalline cellulose and hydroxyalkyl cellulose.

50. A granular dosage form of claim 39,
wherein said at least one second rate-controlling polymer is a combination of microcrystalline cellulose and hydroxypropylmethyl cellulose.

51. A granular dosage form of claim 39,
wherein said at least one second rate-controlling polymer is about 90%, by weight, to about 99.9%, by weight, of microcrystalline cellulose and about 0.1%, by weight, to about 10%, by weight, of hydroxypropylmethyl cellulose, based on the total weight of said at least one second rate-controlling polymer.

52. A kit, comprising:
instructions for administering an oral dosage form to a patient suffering from inflammatory bowel disease;
a granular dosage form of claim 39; and
at least one oral delivery vehicle.

53. A kit, comprising:
instructions for administering an enema to a patient suffering from inflammatory bowel disease;
a granular dosage form of claim 39; and
at least one enema delivery vehicle.
